SEQUENCE LISTING (2 sequences)

SEQ ID NO: 1 - DNA, 20 bp, Artificial Sequence
  Primer CCND2F to amplify a 2.8k bp fragment of CCND2 gene.
atcaccaacacagacgtgga

SEQ ID NO: 2 - DNA, 20 bp, Artificial Sequence
  Primer CCND2R to amplify a 2.8k bp fragment of CCND2 gene.
ttgaaaacccgaccgtttag

(19) United States
(12) Patent Application Publication
(10) Pub. No.: US 2012/0164654 A1
(43) Pub. Date: Jun. 28, 2012

(54) COMPOSITION FOR REVERSE TRANSCRIPTION POLYMERASE CHAIN REACTION

(75) Inventors: Yuko Nakabayashi, Otsu-shi (JP); Yoshimi Sato, Otsu-shi (JP); Takashi Uemori, Otsu-shi (JP); Hiroyuki Mukai, Otsu-shi (JP)

(73) Assignee: TAKARA BIO INC., Otsu-shi (JP)

(21) Appl. No.: 13/393,302

(22) PCT Filed: Aug. 27, 2010
(86) PCT No.: PCT/JP2010/064614

§ 371 Date: Feb. 29, 2012

(30) Foreign Application Priority Data
Sep. 1, 2009 (JP) 2009-201561

(51) Int. Cl. G01N 27/26; C12P 19/34; C12N 9/12

(52) U.S. Cl. 435/6.12; 435/194; 435/91.2

(57) ABSTRACT

A composition for a reverse transcription polymerase chain reaction, which comprises a thermostable DNA polymerase, a reverse transcriptase, a dye marker and a specific gravity-increasing agent; and a premix reagent for a one-step RT-PCR, which comprises the composition, is not frozen under usual storage conditions at −20 to −30° C. and has excellent handleability.

### TECHNICAL FIELD

The present invention relates to a composition useful for a reverse transcription polymerase chain reaction, a method of synthesizing a cDNA using the composition, a method of detecting an RNA using the composition, and a kit for a reverse transcription polymerase chain reaction.

The present application claims the benefit of priority based on Japanese Patent Application No. 2009-201561 filed on Sep. 1, 2009, and the entire contents of Japanese Patent Application No. 2009-201561 are incorporated in the present application.

### BACKGROUND ART

A polymerase chain reaction (PCR) method is a technique for simply amplifying a desired nucleic acid fragment in vitro, and has become an indispensable experimental procedure in not only the field of gene study, but also wide fields of biology, medical science, agriculture and the like. The PCR method is also applied to a method of detecting an RNA, and is referred to as a reverse transcription-polymerase chain reaction or reverse transcriptase-polymerase chain reaction (RT-PCR) method. The RT-PCR method is a method of synthesizing a DNA transcript complementary to an RNA (cDNA) with the use of a reverse transcriptase having RNA-dependent DNA polymerase activity, that is, reverse transcription activity, or a DNA polymerase having reverse transcription activity, and subsequently carrying out PCR using the DNA transcript as a template to specifically amplify and detect the cDNA derived from the RNA. The RT-PCR method is utilized in cloning a cDNA derived from a mRNA and preparing a cDNA library and, additionally, is useful as a method of investigating the expression state of a specific mRNA.

Generally, the RT-PCR method involves a two-step reaction system which comprises first synthesizing a cDNA by a reverse transcription reaction and then amplifying the cDNA by PCR. However, in this method, the more samples, the more labor and time are required and the more likely contaminations between the samples are to occur. As a countermeasure of this problem, a one-step RT-PCR method has been developed, in which a reverse transcription reaction and PCR can be continuously conducted in one container (Non-Patent Literature 1). This method is a system in which a reverse transcriptase and a thermostable DNA polymerase are present in the same reaction system, so that cDNA synthesis using an RNA as a template and PCR amplification using the synthesized cDNA as a template can be controlled by a temperature program.

As a method of analyzing an amplification product obtained by a gene amplification reaction such as RT-PCR, electrophoresis, by which the chain length and amount of the amplification product can be known at the same time, is used frequently. In analysis of the amplification product by electrophoresis, generally, a portion of a solution after reaction is collected, and subjected to electrophoresis after addition of a sample dye buffer (loading buffer). Such a sample dye buffer contains a dye (dye marker) as a component to be an index of a migration distance, and contains glycerol or the like as a component (specific gravity-increasing agent) to increase the specific gravity of a reaction solution so that the reaction solution can be easily applied to wells in a gel.

When a gene amplification method is carried out using a number of samples and the results are analyzed by electrophoresis, work to mix individual reaction solutions with a sample dye buffer and subject the mixture to electrophoresis is very troublesome. This work requires a series of operations such as collection of a reaction solution from a container after completion of an amplification reaction, addition of the dye buffer for a sample, and mixing, and also requires a new container for mixing. Further, there is also a risk that samples may be mistaken during operations. In order to solve this problem, a reaction solution for PCR to which a dye marker and a specific gravity-increasing agent have been added in advance so that the reaction solution after PCR can be directly subjected to electrophoresis has been developed (Patent Literature 1). However, since it is necessary that the dye marker and the specific gravity-increasing agent do not adversely influence on both of different two enzymes, it is believed that development of a reaction solution for one-step RT-PCR to which the dye marker and the specific gravity-increasing agent have been added in advance is difficult.

### CITATION LIST

### Patent Literature

Patent Literature 1: Japanese Patent No. 3587284

### Non-Patent Literature

Non-Patent Literature 1: BioTechniques, vol. 18, No. 4, pp. 678-687 (1995)

### DISCLOSURE OF INVENTION

### Problems to be Solved by the Invention

An object of the present invention is to provide a composition for a reverse transcription polymerase chain reaction comprising a dye marker and a specific gravity-increasing agent, which allows two reactions of a reverse transcription reaction by a reverse transcriptase and PCR by a thermostable DNA polymerase to be effectively carried out.

### Means for Solving the Problems

The present inventors have intensively made efforts in order to solve the above-mentioned problems and, as a result, found that even a composition containing a dye marker and a specific gravity-increasing agent allows one-step RT-PCR excellent in reactivity to be carried out without adversely influencing on both reactions of a reverse transcription reaction by a reverse transcriptase and PCR by a thermostable DNA polymerase, and the reaction solution after RT-PCR can be directly subjected to a gel for electrophoresis. Further, the present inventors have found that the composition can be used to prepare a 5-fold concentration premix reagent for one-step RT-PCR which is not frozen even under a storage condition of −20 to −30° C., a usual condition for storing an enzyme solution and which is excellent in operability. Thus, the present invention has been completed.

That is, a first aspect of the present invention relates to a composition for a reverse transcription polymerase chain reaction comprising a thermostable DNA polymerase, a reverse transcriptase, a dye marker, and a specific gravity-increasing agent. Examples of the specific gravity-increasing agent in the first aspect of the present invention include glycerol, ethylene glycol, polyethylene glycol, and a combination thereof, and examples of the dye marker include Tartrazine, Acid Red 18, Xylene Cyanol and a combination thereof. The composition of the first aspect of the present invention comprises, for example, 20 to 30% by volume of glycerol and 2.5 to 7.5% by weight/volume of polyethylene glycol as the specific gravity-increasing agents, and additionally 5 to 7.5% by volume of ethylene glycol may be contained as the specific gravity-increasing agent.

A second aspect of the present invention relates to a reaction solution for a reverse transcription polymerase chain reaction comprising the composition of the first aspect of the present invention, as well as an RNA to be used as a template, and at least one kind of oligonucleotide primer. The reaction solution of the second aspect of the present invention comprises, for example, 4 to 6% by volume of glycerol and 0.5 to 1.5% by weight/volume of polyethylene glycol as the specific gravity-increasing agents, and additionally 1 to 1.5% by volume of ethylene glycol may be contained as the specific gravity-increasing agent.

A third aspect of the present invention relates to a method of synthesizing a cDNA comprising a step of subjecting the reaction solution of the second aspect of the present invention to a reverse transcription polymerase chain reaction.

A fourth aspect of the present invention relates to a method of detecting an RNA comprising steps of (A) subjecting the reaction solution of the second aspect of the present invention to a reverse transcription polymerase chain reaction, and (B) detecting cDNA amplified in the step (A) by electrophoresis.

A fifth aspect of the present invention relates to a kit for a reverse transcription polymerase chain reaction comprising an enzyme solution containing a thermostable DNA polymerase and a reverse transcriptase, as well as a reaction buffer containing a dye marker and a specific gravity-increasing agent.

### Advantages of the Invention

According to the present invention, there are provided a composition for a reverse transcriptase chain reaction, a method of synthesizing a cDNA using the composition, a method of detecting an RNA using the composition, and a kit for a reverse transcription polymerase chain reaction, which are simple, has low probability in occurrence of contaminations and mistake of samples, and are excellent in reactivity.

### BRIEF DESCRIPTION OF DRAWINGS

FIG. 1 shows agarose gel electrophoresis results of reaction products obtained by using a reaction solution for one-step RT-PCR containing polyethylene glycol as a specific gravity-increasing agent.

FIG. 2 shows agarose gel electrophoresis results of reaction products obtained by using a reaction solution for one-step RT-PCR containing 6% glycerol and various concentrations of polyethylene glycol as a specific gravity-increasing agent.

FIG. 3 shows agarose gel electrophoresis results of reaction products obtained by using a reaction solution for one-step RT-PCR containing various concentrations of glycerol, polyethylene glycol and/or ethylene glycol as a specific gravity-increasing agent.

### MODE FOR CARRYING OUT THE INVENTION

### (1) Composition of the Present Invention

The composition of the present invention comprises a thermostable DNA polymerase, a reverse transcriptase, a dye marker, a specific gravity-increasing agent, and a reaction buffer.

As used herein, the thermostable DNA polymerase refers to a DNA-dependent DNA polymerase retaining activity even after treatment at a temperature of 75° C. or higher for 30 minutes. In the present invention, the thermostable DNA polymerase may further have 5′→3′ exonuclease activity, 3′→5′ exonuclease activity, and/or RNA-dependent DNA polymerase activity.

The thermostable DNA polymerase used in the present invention dose not particularly limit the present invention, and examples thereof include DNA polymerases derived from extreme thermophiles. The extreme thermophile refers to a bacterium viable in an environment of 75° C. or more. Examples of the extreme thermophile include eubacteria of genus Thermus such as Thermus aquaticus, Thermus thermophilus, Thermus flavus, and Thermus filiformis, archaebacteria of genus Pyrococcus such as Pyrococcus furiosus, Pyrococcus woseii, and Pyrococcus horikoshii, and archaebacteria of genus Thermococcus such as Thermococcus litoralis, Thermococcus celler, Thermococcus siculi, Thermococcus sp. KS-1, and Thermococcus kodakaraensis. The thermostable DNA polymerase used in the present invention can be either a naturally-occurring enzyme or a recombinant enzyme, and a thermostable DNA polymerase whose naturally-occurring amino acid sequence is modified in a range of having thermostable DNA polymerase activity can also be used in the present invention.

The composition of the present invention may comprise two or more kinds of thermostable DNA polymerases. Examples of the two or more kinds of thermostable DNA polymerases include combinations of a thermostable DNA polymerase having 3′→5′ exonuclease activity and a thermostable DNA polymerase substantially not having 3′→5′ exonuclease activity. A technique for carrying out PCR using a reaction solution comprising two kinds of thermostable DNA polymerases is known as LA-PCR (Long and Accurate PCR).

The concentration of the thermostable DNA polymerase in the composition of the present invention may be set so that the concentration becomes a concentration suitable for PCR in an RT-PCR reaction solution prepared using the composition. For example, when a DNA synthesis reaction is carried out using 25 μL of a reaction solution containing a DNA polymerase derived from Thermus aquaticus, the amount of the enzyme in the reaction solution may be 0.125 to 5 U. The thermostable DNA polymerase activity described herein is based on the description of commercially available enzymes. For example, when the activity is measured in a reaction solution for measuring activity (25 mM TAPS Buffer (pH 9.3, 25° C.), 50 mM KCl, 2 mM MgCl₂, 1 mM 2-mercaptoethanol, each 200 μM of dATP.dGTP.dTTP, 100 μM [α-32P] dCTP and 0.25 mg/mL activated salmon sperm DNA), using activated salmon sperm DNA as a template/primer at 74° C. for 30 minutes, the activity at which 10 nmol of total nucleotides are incorporated into an acid-insoluble precipitate may be adopted as 1 U.

The reverse transcriptase used in the present invention may be a reverse transcriptase having reverse transcription activity, that is, activity of synthesizing DNA complementary to an RNA as a template, and examples thereof include virus-derived reverse transcriptases such as a Moloney murine leukemia virus-derived reverse transcriptase (MMLV-derived reverse transcriptase) and an avian myeloblastosis virus-derived reverse transcriptase (AMV-derived reverse transcriptase), and eubacterium-derived reverse transcriptases such as DNA polymerases from thermophilic bacteria of the genus Bacillus (Bca DNA polymerase etc.). In the present invention, a virus-derived reverse transcriptase is preferably used, and an MMLV-derived reverse transcriptase is more preferably used. The reverse transcriptase used in the present invention can be a naturally-occurring enzyme or a recombinant enzyme, and a reverse transcriptase whose naturally-occurring amino acid sequence is modified in a range of having reverse transcription activity can also be used in the present invention.

The concentration of the reverse transcriptase in the composition of the present invention may be set so that the concentration becomes a concentration suitable for a reverse transcription reaction in an RT-PCR reaction solution prepared using the composition, for example, the concentration of the reverse transcriptase in a reaction solution when RT-PCR is carried out becomes the concentration of the reverse transcriptase used in the conventional one-step RT-PCR. For example, when one-step RT-PCR is carried out using 50 μL of a reaction solution containing an MMLV-derived reverse transcriptase, the amount of the enzyme in the reaction solution may be 1 to 200 U, and is preferably 5 U to 60 U, further preferably 10 to 30 U from the viewpoint of cDNA synthesis efficiency. The reverse transcriptase activity described herein is based on the description of commercially available enzymes. For example, the enzyme activity at which 1 nmol of [³H] dTTP is incorporated into an acid-insoluble precipitate at 37° C. for 10 minutes using Poly(rA).oligo(dT)₁₂₋₁₈ as a template/primer, may be adopted as 1 U.

The dye marker used in the present invention can be utilized as an index of a migration distance at electrophoresis after RT-PCR, and is not particularly limited as far as it does not inhibit the reverse transcription reaction by a reverse transcriptase and the PCR by a thermostable DNA polymerase. Examples of the dye marker that can be utilized as an index of a migration distance at electrophoresis include Tartrazine, Orange G, Orange GII, Bromo Phenol Blue, Acid Blue 9, Ponceau S, Acid Red 18, Xylene Cyanol, Fast Green FCF, and Amido Black. The composition of the present invention may comprise only one kind of dye marker, or two or more kinds of dye markers. When two kinds of dye markers are contained in the composition of the present invention, preferable examples of combinations of the dye markers, which do not particularly limit the present invention, include a combination of Xylene Cyanol and Tartrazine and a combination of Xylene Cyanol and Acid Red 18.

The concentration of the dye marker in the composition of the present invention may be a concentration which does not inhibit the reverse transcription reaction and the PCR in the reaction solution and is visible as an index of a migration distance at electrophoresis. For example, the concentration of the dye marker in an RT-PCR reaction solution prepared using the composition of the present invention is suitably set to 10 to 250 ng/μL in the case of Xylene Cyanol, 25 to 100 ng/μL in the case of Orange GII, or 25 to 50 ng/μL in the case of Ponceau S.

The specific gravity-increasing agent used in the present invention refers to a compound which can increase the specific gravity of a reaction solution so that the reaction solution after RT-PCR can be easily added to wells in a gel. Examples of the specific gravity-increasing agent include glycerol, ethylene glycol (EG), polyethylene glycol (PEG), and sucrose. The concentration of the specific gravity-increasing agent in the reaction solution is not particularly limited as long as it is in such a range that the agent does not adversely influence on the reverse transcription reaction by a reverse transcriptase and the PCR by a thermostable DNA polymerase and provides suitable specific gravity to the reaction solution. For example, the concentration of the specific gravity-increasing agent in the reaction solution when RT-PCR is carried out is suitably set to 2 to 6% by volume (v/v %) in the case of glycerol, 0.5 to 10% by weight/volume (w/v %) in the case of polyethylene glycol, or 1 to 3 v/v % in the case of ethylene glycol. In the case where the concentration of polyethylene glycol is 3 to 10 w/v % in the reaction solution when RT-PCR is carried out, the amplified amount of the reaction product is large. Therefore, it is preferable that the composition of the present invention is designed so as to prepare a reaction solution containing the specific gravity-increasing agent at such a concentration as described above.

The composition of the present invention may comprise only one kind of specific gravity-increasing agent, or two or more kinds of specific gravity-increasing agents. When two or more kinds of specific gravity-increasing agents are contained in the composition of the present invention, preferable examples of combinations of specific gravity-increasing agents, which do not particularly limit the present invention, include a combination of two or more kinds of specific gravity-increasing agents selected from the group consisting of glycerol, polyethylene glycol, and ethylene glycol. For example, since a reaction solution for one-step RT-PCR comprising 6 v/v % glycerol and 0.5 to 2 w/v % polyethylene glycol as the specific gravity-increasing agents provides a larger amplified amount of the reaction product as compared with the case where a reaction solution comprising only 6 v/v % glycerol as the specific gravity-increasing agent, a composition suitable for preparation of the reaction solution is suitable in the present invention. Examples of a suitable aspect of the present invention include a 5-fold concentration premix reagent for one-step RT-PCR comprising 30 v/v % glycerol and 2.5 to 7.5 w/v % polyethylene glycol as the specific gravity-increasing agents, and a 5-fold concentration premix reagent for one-step RT-PCT comprising 20 to 30 v/v % glycerol, 2.5 to 7.5 w/v % polyethylene glycol, and 5 to 7.5 v/v % ethylene glycol as the specific gravity-increasing agent. Since these premix reagents are not frozen even under a storage condition of −20 to −30° C. and exhibit excellent reactivity when utilized in RT-PCR, they are suitable as the composition of the present invention.

The composition of the present invention may further comprise a reaction buffer, at least one kind of deoxyribonucleotide, a magnesium salt and/or a manganese salt.

A reaction buffer refers to a compound or a mixture capable of mitigating variation in the hydrogen ion concentration (pH) of a reaction solution. Generally, a mixed solution of a weak acid or a salt thereof, or a weak base or a salt thereof is widely used as the reaction buffer for the purpose of pH control because it has strong buffering action. The pH of the composition of the present invention is suitably set in a usual range where a gene amplification reaction is carried out, for example, in a range of pH 8.3 to pH 9.5.

A deoxyribonucleotide is composed of a deoxyribose bonded to an organic base to which a phosphate group is bonded via a phosphoester bond. A naturally-occurring DNA contains four different nucleotides. A nucleotide having adenine, guanine, cytosine or thymine base is seen in a naturally-occurring DNA. Adenine, guanine, cytosine and thymine bases are often abbreviated as A, G, C and T, respectively.

The deoxyribonucleotide used in the present invention is a free triphosphate-type (that is, a phosphate group has three phosphate parts) deoxyribonucleotide, that is, deoxyribonucleoside triphosphate (e.g. dATP, dCTP, dITP, dGTP and dTTP) and, further, derivatives thereof can be also used. The deoxyribonucleotide derivative includes [αS]dATP, 7-deaza-dGTP, 7-deaza-dATP, and deoxynucleotide derivatives exhibiting resistance to nucleic acid degradation. The nucleotide derivative includes a deoxyribonucleotide being labeled with a radioactive isotope such as ³²P or ³⁵S, a fluorescent part, a chemiluminescent part, a bioluminescent part or an enzyme in order to allow for detection.

The composition of the present invention containing a reaction buffer, a deoxyribonucleotide, as well as a magnesium salt and/or a manganese salt can be formulated into a reaction solution for RT-PCR only by adding an RNA to be used as a template, an oligonucleotide primer and, if necessary, water, before implementation of RT-PCR. That is, a premix reagent for one-step RT-PCR is one of aspects of the composition of the present invention. In this case, for example, a reaction solution for one-step RT-PCR can be prepared by adding an RNA to be used as a template, an oligonucleotide primer and, if necessary, water to the composition of the present invention to dilute the composition of the present invention 1.5 to 5-fold. Since a premix reagent requiring 6-fold or more dilution causes a too large measuring error when it is used to prepare a reaction solution, it is not preferable as an aspect of the present invention.

Preferably, the premix reagent for one-step RT-PCR is not frozen by storage at −20 to −30° C. which is a usual storage temperature for an enzyme solution. If the premix reagent is not frozen under the storage conditions, operations of melting and mixing are not required at the time of using the premix reagent, and inactivation of an enzyme due to repetition of freezing and melting can be avoided.

For example, since the premix reagent for one-step RT-PCR comprising 30 v/v % glycerol and 2.5 to 7.5 w/v % polyethylene glycol as the specific gravity-increasing agent, ands the premix reagent for one-step RT-PCR comprising 20 to 30 v/v % glycerol, 2.5 to 7.5 w/v % polyethylene glycol, and 5 to 1.5 v/v % ethylene glycol as the specific gravity-increasing agent are not frozen even under a storage condition of −20 to −30° C., and exhibit excellent reactivity when they are utilized in RT-PCR, they are preferable as the composition of the present invention.

In addition, a reaction solution for RT-PCR prepared using the above-mentioned premix reagent is included in the present invention. The reaction solution of the present invention can be prepared using the composition of the present invention, an RNA to be used as a template, and at least one kind of oligonucleotide primer.

The RNA to be used as a template is an RNA which can serve as a template of a reverse transcription reaction from a primer when the primer is hybridized with the RNA. The composition of the present invention may comprise one kind of template, or plural kinds of templates having different nucleotide sequences. By using a primer specific for a particular template, primer extension products can be produced based on plural kinds of templates in a mixture of nucleic acids. The plural templates may be present in different nucleic acids, or may be present in the same nucleic acid.

Examples of the RNA used as a template and which can be applied to the present invention include, but are not particularly limited to, an RNA molecule group such as total RNA, mRNA, tRNA, and rRNA in a sample, and a particular RNA molecule group (e.g. an RNA molecule group having a common nucleotide sequence motif, products of transcription by an RNA polymerase, an RNA molecule group concentrated by a subtraction method), and further include any RNA from which a primer used in a reverse transcription reaction can be prepared.

In the present invention, the RNA to be used as a template may be contained in, for example, a sample derived from a living body, such as cell, tissue or blood; or a sample that may contain an organism, such as food, soil or waste water; or the like, or the RNA may be contained in a nucleic acid-containing preparation obtained by treating the sample or the like with a known method. Examples of the preparation include a cell disruption product and a sample obtained by fractionation thereof, total RNA in the sample, and a sample enriched in a particular RNA molecule group, for example, mRNA.

The oligonucleotide primer is an oligonucleotide having a base sequence complementary to a template RNA or an oligonucleotide having a base sequence complementary to a cDNA synthesized from a template RNA, and is not particularly limited as long as it anneals an RNA or a cDNA used as a template under reaction conditions to be used. In addition, an oligonucleotide such as oligo (dT) and an oligonucleotide having a random sequence (random primer) can be also utilized in the present invention as the oligonucleotide primer. The chain length of the primer is preferably 6 nucleotides or more, further preferably 10 nucleotides or more from the viewpoint of implementation of specific annealing. The chain length of the primer is preferably 100 nucleotides or less, further preferably 30 nucleotides or less from the viewpoint of synthesis of an oligonucleotide. The above-mentioned oligonucleotide can be chemically synthesized, for example, by a known method. In addition, the oligonucleotide may be an oligonucleotide derived from an organism sample and, for example, it may be prepared by isolation from a restriction endonuclease digestion product of a DNA prepared from a natural sample.

The concentration of the oligonucleotide primer in the composition of the present invention may be set so that the concentration of the oligonucleotide primer in a reaction solution at the time of carrying out RT-PCR becomes a concentration suitable for one-step RT-PCR, and may be a concentration of 0.1 to 1 μM without particular limitation.

(2) Method of Synthesizing a cDNA of the Present Invention

The method of synthesizing a cDNA of the present invention comprises a step of subjecting the reaction solution for RT-PCR of the present invention to a reverse transcription polymerase chain reaction. The reverse transcription polymerase chain reaction is preferably a one-step reverse transcription polymerase chain reaction in which a polymerase chain reaction is directly carried out after completion of a reverse transcription reaction.

The conditions of the reverse transcription reaction in the method of synthesizing a cDNA of the present invention are not particularly limited as long as they are sufficient conditions for synthesizing a primer extension chain complementary to a template RNA. The sufficient conditions for synthesizing a primer extension chain complementary to a template RNA are not particularly limited, and examples of the temperature condition include 25 to 60° C., and 30 to 50° C. is more preferable. In addition, examples of the reaction time include 5 to 120 minutes, and 15 to 60 minutes is more preferable. The reverse transcription reaction in one-step RT-PCR may be carried out at a higher temperature than that of a general reverse transcription reaction [BioTechniques, vol. 18, No. 4, pp. 678-pp. 687 (1995)]. In the case of the reverse transcription reaction at a higher temperature than that of a general reverse transcription reaction, a reverse transcriptase is inactivated with passage of the reaction time and, on the other hand, the specificity of annealing of a primer is improved and, moreover, influence due to the secondary structure of an RNA to be used as a template can be avoided and, as a result, the specificity of the reverse transcription reaction and the reaction sensitivity are improved. For example, a general reverse transcription reaction temperature for a reverse transcription reaction using a reverse transcriptase derived from MMLV is 42° C., and an optimal temperature at the reaction initial velocity of this reverse transcriptase is 44 to 50° C. [J. Biochem., vol. 143, pp. 261-pp. 268 (2008)], but for the purpose of improving the sensitivity and specificity of RT-PCR, the reverse transcription reaction may be carried out at 45 to 50° C., or at a temperature of 50° C. or higher which is higher than the optimal temperature of an MMLV-derived reverse transcriptase.

In the method of synthesizing a cDNA of the present invention, after the above-mentioned reverse transcription reaction, the reaction solution may be incubated at conditions under which the reverse transcriptase is inactivated. Examples of the conditions under which the reverse transcriptase is inactivated include conditions in which the reaction solution is incubated at 94° C. for 2 minutes.

The conditions of the polymerase chain reaction in the method of synthesizing a cDNA of the present invention may be general PCR conditions. For example, the polymerase chain reaction is carried out by a reaction consisting of three steps of dissociation (denaturation) of a double-stranded template DNA into a single strand, annealing of a primer to the single-stranded template DNA and complementary strand synthesis (extension) from the primer, or a two-step reaction called “shuttle PCR” [‘PCR Method Frontier’, “Protein, nucleic acid and enzyme” extra issue, vol. 41, No. 5, pp. 425-pp. 428 (1996)], in which the steps of primer annealing and extension among the above-mentioned three-step reaction are carried out at the same temperature.

### (3) Method of Detecting an RNA of the Present Invention

The method of detecting an RNA of the present invention comprises steps of (A) subjecting the reaction solution for RT-PCR of the present invention to a reverse transcription polymerase chain reaction, and (B) detecting a cDNA amplified in the step (A) by electrophoresis. The reverse transcription polymerase chain reaction in the step (A) can be carried out under the same conditions as those of “(2) Method of synthesizing a cDNA of the present invention”.

Examples of the electrophoresis in the step (B) include agarose gel electrophoresis and polyacrylamide gel electrophoresis. As a buffer for running used in electrophoresis (running buffer), in the case of the agarose gel electrophoresis, a TAE buffer or a TBE buffer is generally used and, in the case of the polyacrylamide gel electrophoresis, a TBE buffer is generally used. In the case of the agarose gel electrophoresis, the TAE buffer is excellent in separation of a long chain DNA of a few kb or more, and the TBE buffer is excellent in separation of a short chain DNA. The reaction solution after RT-PCR carried out in the step (A) can be subjected to electrophoresis as it is without adding a dye buffer for a sample, in the case of using either the TAE buffer or the TBE buffer as the running buffer.

### (4) Kit for a Reverse Transcription Polymerase Chain Reaction of the Present Invention

The kit for a reverse transcription polymerase chain reaction of the present invention comprises an enzyme solution containing a thermostable DNA polymerase and a reverse transcriptase, as well as a reaction buffer containing a dye marker and a specific gravity-increasing agent.

As the thermostable DNA polymerase and the reverse transcriptase contained in the enzyme solution, and the dye marker and the specific gravity-increasing agent contained in the reaction buffer, those exemplified in “(1) Composition of present invention” can be suitably utilized. The specific gravity-increasing agent may be contained in not only the reaction buffer, but also the enzyme solution.

The reaction buffer containing the dye marker and the specific gravity-increasing agent may further contain the reaction buffer, at least one kind of deoxyribonucleotide, magnesium salt and/or manganese salt exemplified in “(1) Composition of the present invention”.

A reaction solution for a reverse transcription polymerase chain reaction can be prepared by mixing the enzyme solution and the reaction buffer contained in the kit of the present invention, and further adding an RNA to be used as a template, an oligonucleotide primer and, if necessary, water to the mixture.

### EXAMPLES

### Example 1

Addition of a dye marker and a specific gravity-increasing agent to a reaction solution for one-step RT-PCR was studied. For preparing the reaction solution for RT-PCR, PrimeScript (registered trademark) 1 step Enzyme Mix contained in PrimeScript (registered trademark) One Step RT-PCR Kit Ver. 2 (manufactured by TAKARA BIO INC.) was used as a reverse transcriptase and a thermostable DNA polymerase. As a template, total RNA obtained from a HL60 cell was used. As a primer, a primer pair of CCND2F (SEQ ID No.: 1) and CCND2R (SEQ ID No.: 2) for amplifying a 2.8 kb region of a CCND2 gene was used.

First, five kinds of 25 μL of reaction solutions (containing 50 pg, 500 pg, 5 ng, 50 ng and 500 ng of a template total RNA, respectively) to which a dye marker and a specific gravity-increasing agent were not added were prepared, as controls. For preparing the control reaction solutions, 1 μL of PrimeScript (registered trademark) 1 step Enzyme Mix contained in PrimeScript (registered trademark) One Step RT-PCR Kit Ver. 2 was used as a reverse transcriptase and a thermostable DNA polymerase. Further, the composition of the reaction solution other than the enzyme and template total RNA comprised 50 mM Tris-HCl (pH 9.2), 2.5 mM MgCl₂, 14 mM (NH₄)₂SO₄, each 0.4 mM dNTP, 12.5 mM KCl, 0.01% BSA, 400 nM CCDN2F, and 400 nM CCDN2R. The PrimeScript (registered trademark) 1 step Enzyme Mix is an enzyme mixed solution containing PrimeScript (registered trademark) RTase which is an MMLV-derived reverse transcriptase, and TaKaRa Ex Taq (registered trademark) HS which is a mixture of two kinds of thermostable DNA polymerases. The reaction solution contains glycerol at a final concentration of 2 v/v %, which is brought from PrimeScript (registered trademark) 1 step Enzyme Mix.

Then, 25 μL of the reaction solutions 1 to 7 each containing Xylene Cyanol FF (manufactured by NACALAI TESQUE INC.) or Tartrazine (manufactured by NACALAI TESQUE INC.) as a dye marker, and containing glycerol as a specific gravity-increasing agent were prepared for 50 pg, 500 pg, 5 ng, 50 ng and 500 ng of a template total RNA respectively. The reaction solutions 1 to 7 are different in the glycerol concentration, as shown in Table 1. In addition, the reaction solutions 1 to 7 for each template total RNA amount contain the same components as those of the above-mentioned control reaction solution except that they contain a dye marker and glycerol at concentrations shown in Table 1.

TABLE 1

| | Glycerol | Xylene Cyanol FF | Tartrazine |
|---|---|---|---|
| | (v/v %) | (ng/μL) | (ng/μL) |
| Control | 2 | — | — |
| Reaction | 2 | 25 | 250 |
| solution 1 | | | |
| Reaction | 4 | 25 | 250 |
| solution 2 | | | |
| Reaction | 6 | 25 | 250 |
| solution 3 | | | |
| Reaction | 8 | 25 | 250 |
| solution 4 | | | |
| Reaction | 10 | 25 | 250 |
| solution 5 | | | |
| Reaction | 15 | 25 | 250 |
| solution 6 | | | |
| Reaction | 20 | 25 | 250 |
| solution 7 | | | |

Using each 25 μL of the prepared reaction solutions, one-step RT-PCR was carried out using TaKaRa PCR Thermal Cycler Dice (registered trademark, manufactured by TAKARA BIO INC.). One-step RT-PCR was carried out by reverse transcription reaction at 42° C. for 30 minutes, subsequently heating at 94° C. for 2 minutes, and then PCR of a total of 30 cycles in which incubation at 98° C. for 10 seconds, 55° C. for 30 seconds and 72° C. for 3 minutes constitute one cycle. Three μL was collected from each reaction solution after completion of the reaction, and subjected to 1% agarose electrophoresis, and the amount of an amplification product was confirmed by UV irradiation.

As a result, it was made clear that even a reaction solution containing a dye marker and a specific gravity-increasing agent allowed a cDNA to be amplified from a template RNA by one-step RT-PCR. In addition, the reaction solution containing dye markers and glycerol at a final concentration of 2 v/v % or more could be directly applied to an agarose gel without adding a sample dye buffer after completion of the reaction, even when either a TAE buffer or a TBE buffer was used as a running buffer for agarose electrophoresis. In the reaction solution containing dye markers and glycerol at a concentration of 4 v/v % or less as a specific gravity-increasing agent, both the sensitivity and the amount of amplification were equal to those in the control. However, in the reaction solution containing dye markers and glycerol at 6 v/v % as a specific gravity-increasing agent, the amount of amplification was slightly reduced as compared with the control. In the reaction solution containing dye markers and glycerol at a concentration of 8 v/v % or more as a specific gravity-increasing agent, both the sensitivity and the amount of amplification were reduced.

### Example 2

For a reaction solution for one-step RT-PCR containing a dye marker and a specific gravity-increasing agent, an effect of use of polyethylene glycol as the specific gravity-increasing agent was studied.

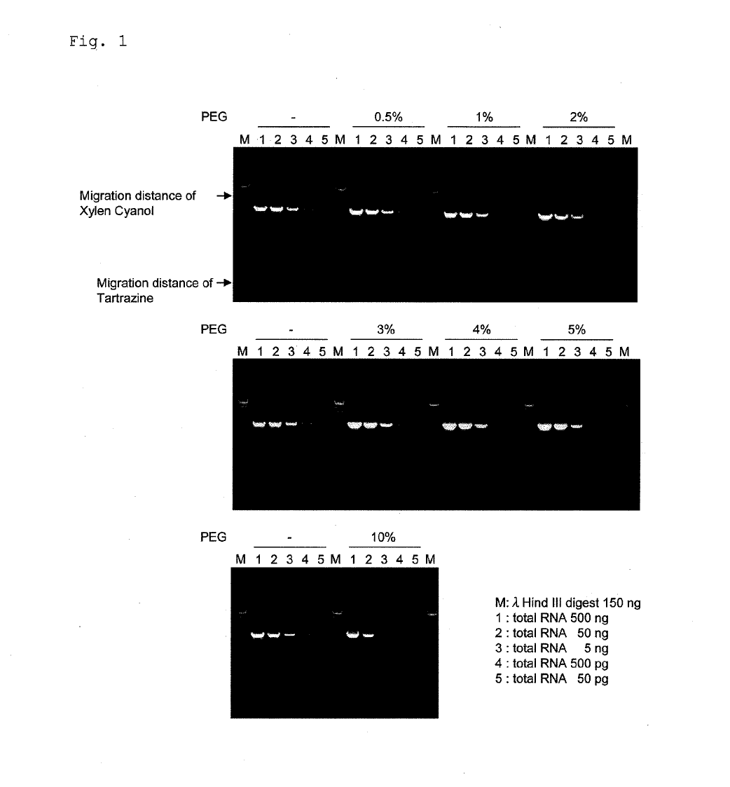

Reaction solutions for RT-PCR were prepared in the same manner as in Example 1 except that reaction solutions 1 to 7 containing polyethylene glycol #6000 (manufactured by NACALAI TESQUE INC.; in the following examples, simply described as polyethylene glycol or PEG) at concentrations described in Table 2 as the specific gravity-increasing agent were prepared. Using each prepared reaction solution, one-step RT-PCR was carried out in the same manner as in Example 1. The results of agarose electrophoresis are shown in FIG. 1.

TABLE 2

| | Glycerol | | Xylene Cyanol FF | Tartrazine |
|---|---|---|---|---|
| | (v/v %) | PEG (w/v %) | (ng/μL) | (ng/μL) |
| Control | 2 | — | — | — |
| Reaction | 2 | 0.5 | 25 | 250 |
| solution 1 | | | | |
| Reaction | 2 | 1 | 25 | 250 |
| solution 2 | | | | |
| Reaction | 2 | 2 | 25 | 250 |
| solution 3 | | | | |
| Reaction | 2 | 3 | 25 | 250 |
| solution 4 | | | | |
| Reaction | 2 | 4 | 25 | 250 |
| solution 5 | | | | |
| Reaction | 2 | 5 | 25 | 250 |
| solution 6 | | | | |
| Reaction | 2 | 10 | 25 | 250 |
| solution 7 | | | | |

As a result, it was made clear that even a dye marker-containing reaction solution containing polyethylene glycol as a specific gravity-increasing agent allowed a cDNA to be amplified from a template RNA by one-step RT-PCR. In addition, the reaction solution containing dye markers and polyethylene glycol at a final concentration of 0.5 w/v % or more could be directly applied to an agarose gel without adding a sample dye buffer after completion of the reaction, even when either a TAE buffer or a TBE buffer was used as a running buffer for agarose electrophoresis. In the reaction solution containing 3 to 10 w/v % polyethylene glycol as the specific gravity-increasing agent, the amount of amplification was increased as compared with the control. In addition, in the reaction solution containing 0.5 to 5 w/v % polyethylene glycol, the detection sensitivity was equal to that of the control.

### Example 3

For a reaction solution for one-step RT-PCR containing a dye marker and a specific gravity-increasing agent, an effect of use of ethylene glycol as the specific gravity-increasing agent was studied.

Reaction solutions for RT-PCR were prepared in the same manner as in Example 1 except that reaction solutions 1 to 7 containing ethylene glycol (manufactured by NACALAI TESQUE INC.) at each concentration described in Table 3 as the specific gravity-increasing agent were prepared. Using each prepared reaction solution, one-step RTP-PCR was carried out in the same manner as in Example 1. In Table 3, EG means ethylene glycol.

TABLE 3

| | | | Xylene | |
|---|---|---|---|---|
| | Glycerol | EG | Cyanol FF | Tartrazine |
| | (v/v %) | (v/v %) | (ng/μL) | (ng/μL) |
| Control | 2 | — | — | — |
| Reaction | 2 | 1 | 25 | 250 |
| solution 1 | | | | |
| Reaction | 2 | 2 | 25 | 250 |
| solution 2 | | | | |
| Reaction | 2 | 3 | 25 | 250 |
| solution 3 | | | | |
| Reaction | 2 | 4 | 25 | 250 |
| solution 4 | | | | |
| Reaction | 2 | 5 | 25 | 250 |
| solution 5 | | | | |
| Reaction | 2 | 6 | 25 | 250 |
| solution 6 | | | | |
| Reaction | 2 | 8 | 25 | 250 |
| solution 7 | | | | |

As a result, it was made clear that even a dye marker-containing reaction solution containing ethylene glycol as a specific gravity-increasing agent allowed a cDNA to be amplified from a template RNA by one-step RT-PCR. In addition, the reaction solution containing dye markers and ethylene glycol at a final concentration of 1 v/v % or more could be directly applied to an agarose gel without adding a sample dye buffer after completion of the reaction, when either a TAE buffer or a TBE buffer was used as a running buffer for agarose electrophoresis. In the reaction solution containing 1 v/v % ethylene glycol as a specific gravity-increasing agent, the amount of amplification was equal to that of the control. In addition, in the reaction solution containing 1 to 3 v/v % ethylene glycol as a specific gravity-increasing agent, the detection sensitivity was equal to that of the control.

### Example 4

Influence of various dye markers on 1 step RT-PCR was studied.

Reaction solutions for one-step RT-PCR were prepared in the same manner as in Example 1 except that the reaction solutions contained, as a dye marker, Orange G (manufactured by NACALAI TESQUE INC.), Orange GII (manufactured by NACALAI TESQUE INC.), Bromo Phenol Blue (manufactured by NACALAI TESQUE INC.), Acid Blue 9 (manufactured by Merck), Amaranth (manufactured by NACALAI TESQUE INC.), Acid Red 18 (manufactured by Tokyo Chemical Industry Co., Ltd.), or Ponceau S (manufactured by Merck) at concentrations described in Table 4 in place of Xylene Cyanol FF and Tartrazine, and did not contain a specific gravity-increasing agent other than glycerol at a final concentration of 2 v/v % which was brought from PrimeScript (registered trademark) 1 step Enzyme Mix. Using each prepared reaction solution, one-step RT-PCR was carried out in the same manner as in Example 1.

TABLE 4

| | | | Concentration of |
|---|---|---|---|
| | Glycerol | | dye marker |
| | (v/v %) | Dye marker | (ng/μL) |
| Control | 2 | — | — |
| Reaction | 2 | Orange G | 200 |
| solution 1 | | | |
| Reaction | 2 | Orange GII | 100 |
| solution 2 | | | |
| Reaction | 2 | Bromo Phenol | 100 |
| solution 3 | | Blue | |
| Reaction | 2 | Acid Blue 9 | 50 |
| solution 4 | | | |
| Reaction | 2 | Amaranth | 250 |
| solution 5 | | | |
| Reaction | 2 | Acid Red 18 | 250 |
| solution 6 | | | |
| Reaction | 2 | Ponceau S | 50 |
| solution 7 | | | |

As a result, it was made clear that a cDNA could be amplified from a template RNA by one-step RT-PCR, using the reaction solution containing any dye marker. In addition, in the reaction solution containing any dye marker, the detection sensitivity and the amount of amplification were equal to those of the control reaction solution.

### Example 5

Influence of the content of a dye marker on one-step RT-PCR was studied.

Reaction solutions for RT-PCR were prepared in the same manner as in Example 1 except that the reaction solutions contained, as a dye marker, Xylene Cyanol FF, Orange GII, or Ponceau S at concentrations described in Table 5 in place of Xylene Cyanol FF and Tartrazine, and did not contain a specific gravity-increasing agent other than glycerol at a final concentration of 2 v/v % which was brought from PrimeScript (registered trademark) 1 step Enzyme Mix. Regarding each prepared reaction solution, one-step RT-PCR was carried out in the same manner as in Example 1.

TABLE 5

| | | | Concentration |
|---|---|---|---|
| | Glycerol | | of dye marker |
| | (v/v %) | Dye marker | (ng/μL) |
| Control | 2 | — | — |
| Reaction | 2 | Xylene Cyanol | 10 |
| solution 1 | | | |
| Reaction | 2 | Xylene Cyanol | 20 |
| solution 2 | | | |
| Reaction | 2 | Xylene Cyanol | 50 |
| solution 3 | | | |
| Reaction | 2 | Xylene Cyanol | 75 |
| solution 4 | | | |
| Reaction | 2 | Xylene Cyanol | 100 |
| solution 5 | | | |
| Reaction | 2 | Xylene Cyanol | 250 |
| solution 6 | | | |
| Reaction | 2 | Orange GII | 25 |
| solution 7 | | | |
| Reaction | 2 | Orange GII | 50 |
| solution 8 | | | |
| Reaction | 2 | Orange GII | 100 |
| solution 9 | | | |
| Reaction | 2 | Orange GII | 250 |
| solution 10 | | | |
| Reaction | 2 | Ponceau S | 25 |
| solution 11 | | | |
| Reaction | 2 | Ponceau S | 50 |
| solution 12 | | | |
| Reaction | 2 | Ponceau S | 100 |
| solution 13 | | | |
| Reaction | 2 | Ponceau S | 250 |
| solution 14 | | | |

In the reaction solution containing Xylene Cyanol at any concentration of 10 to 250 ng/μL, both the detection sensitivity and the amount of amplification were equal to those of the control. On the other hand, in the reaction solution containing Orange GII at 25 to 100 ng/μL, both the detection sensitivity and the amount of amplification were equal to those of the control, whereas in the reaction solution containing Orange GII at 250 ng/μL, both the detection sensitivity and the amount of amplification were reduced as compared with the control. In the reaction solution containing Ponceau S at 25 to 50 ng/μL, both the detection sensitivity and the amount of amplification were equal to those of the control, whereas in the reaction solution containing Ponceau S at 100 ng/μL or more, both the detection sensitivity and the amount of amplification were reduced as compared with the control.

### Example 6

A 5-fold concentration premix reagent for one-step RT-PCR, which was not frozen even under a storage condition of −30° C. and was excellent in operability, was studied.

### (1) Study of Glycerol Concentration

Each 2 mL of 5-fold concentration premix reagents for one-step RT-PCR (hereinafter, referred to as “5×premix”) containing 10 v/v %, 15 v/v %, 20 v/v %, 25 v/v % and 30 v/v % glycerol were prepared. Components other than glycerol of the 5×premix are shown in Table 6.

TABLE 6

| Component | Concentration | |
|---|---|---|
| PrimeScript (registered trademark) RTase | 2 | U/μL |
| Recombinant RNase Inhibitor | 2 | U/μL |
| Ex Tag Hot Start version | 0.5 | U/μL |
| Tris HCl (pH 9.2) | 253 | mM |
| KCl | 62.5 | mM |
| MgCl₂ | 12.5 | mM |
| (NH₄)₂SO₄ | 70 | mM |
| NaCl | 5 | mM |
| EDTA | 0.06 | mM |
| DTT | 1.3 | mM |
| HEPES NaOH (pH 7.5) | 1 | mM |
| Bovine serum albmin | 0.05 | v/w % |
| Tween20 | 0.15 | v/v % |
| Nonidet P-40 | 0.05 | v/v % |
| dATP | 2 | mM |
| dGTP | 2 | mM |
| dCTP | 2 | mM |
| dTTP | 2 | mM |
| Acid Red 18 | 0.75 | μg/μL |
| Xylen Cyanol FF | 0.125 | μg/μL |

After each 650 μL of the prepared 5×premixes were dispensed into 1.5 mL tubes and allowed to stand for 1 to 2 days in a refrigerator [MEDICAL FREEZER (manufactured by SANYO Electric Co., Ltd.)] set at −30° C., the presence or absence of freezing was confirmed. The results are shown in Table 7.

TABLE 7

| | | | | | |
|---|---|---|---|---|---|
| Glycerol concentration | 2% | 3% | 4% | 5% | 6% |
| (at the time of RT-PCR | | | | | |
| reaction) | | | | | |
| Glycerol concentration | 10% | 15% | 20% | 25% | 30% |
| (5 x premix) | | | | | |
| Presence or absence of | ▪ | ▪ | ▪ | ▪ | □ |
| freezing at −30° C. | | | | | |

In the table, ▪ indicates that a sample was frozen at −30° C., and □ indicates that a sample was not frozen.

As shown in Table 7, the 5×premix containing glycerol at 30 v/v % (glycerol concentration at the time of RT-PCR: 6 v/v %) was not frozen in storage at −30° C.

### (2) Study of Polyethylene Glycol Concentration

A 5-fold concentration premix reagent for one-step RT-PCR, which was not frozen even under a storage condition of −30° C. and was excellent in operability, was studied.

Each 2 mL of a 5×premix containing 30 v/v % glycerol and the components shown in Table 6, as well as 4 kinds of 5×premixes containing 2.5 w/v %, 5 w/v %, 7.5 w/v %, and 10 w/v % polyethylene glycol in addition to 30 v/v % glycerol and the components shown in Table 6 were prepared. As a control, 2 mL of a 5×premix having 2 v/v % glycerol and the composition shown in Table 6 was prepared.

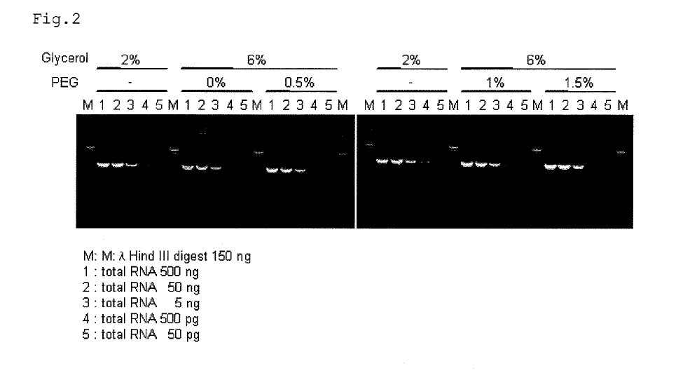

After each 650 μL of the prepared six kinds of 5×premixes were dispensed into 1.5 mL tubes, and allowed to stand for 1 to 2 days in a refrigerator (MEDICAL FREEZER) set at −30° C., the presence or absence of freezing was confirmed. The control 5×premix was frozen, whereas the other five kinds of 5×premixes were not frozen. Then, to each 5 μL of the 5×premixes were added 0.5 μL of 20 μM CCDN2F, 0.5 μL of 20 μM CCDN2R, 1 μL of a template total RNA solution obtained from HL60, and 18 μL of sterilized distilled water to prepare RT-PCR reaction solutions. At this time, as the template total RNA solution, the solutions of 50 pg/μL, 500 pg/μL, 5 ng/μL, 50 ng/μL, and 500 ng/μL were used for each 5×premix, and five kinds of reaction solutions containing different amounts of the template were prepared. In the prepared reaction solutions, one-step RT-PCR was carried out using TaKaRa PCR Thermal Cycler Dice (registered trademark). One-step RT-PCR was carried out by reverse transcription reaction at 42° C. for 30 minutes, subsequently heating at 94° C. for 2 minutes, and then PCR of a total of 30 cycles in which incubation at 98° C. for 10 seconds, 55° C. for 30 seconds and 72° C. for 3 minutes constitute one cycle. Three μL was collected from each reaction solution after completion of the reaction, and subjected to 1% agarose electrophoresis, and the amount of an amplification product was confirmed by UV irradiation. The results are shown in Table 9. In addition, the meanings of symbols described in columns of operability, reactivity and comprehensive evaluation in Table 9 are indicated in Table 8. The results of agarose electrophoresis are shown in FIG. 2.

TABLE 8

| Symbol | Evaluation |
|---|---|
| ⊙ | Excellent |
| ◯ | Normal |
| Δ | Slightly inferior |
| X | Inferior |

TABLE 9

| | | Presence or | | | Compre- |
|---|---|---|---|---|---|
| | | absence of | Oper- | Reactiv- | hensive |
| Glycerol | PEG | freezing | ability | ity | evaluation |
| 6 (v/v) % | 0 (w/v) % | □ | ⊙ | Δ | ◯ |
| | 0.5 (w/v) % | □ | ⊙ | ◯ | ⊙ |
| | 1 (w/v) % | □ | ⊙ | ⊙ | ⊙ |
| | 1.5 (w/v) % | □ | ⊙ | ⊙ | ⊙ |
| | 2 (w/v) % | □ | Δ | ⊙ | ◯ |

In the table, ▪ indicates that a sample was frozen at −30° C., and □ indicates that a sample was not frozen.

In Table 9, the concentration of glycerol and the concentration of polyethylene glycol indicate the concentrations in the reaction solution for RT-PCR which was subjected to the reaction. As shown in Table 9, in RT-PCR using the reaction solution having a glycerol concentration of 6 v/v %, it was made clear that reactivity was improved by further adding 0.5 to 2 w/v % polyethylene glycol to the reaction solution. However, the 5×premix containing 10 w/v % polyethylene glycol (polyethylene glycol concentration at the time of RT-PCR reaction: 2 w/v %) had very high viscosity in the state of 5×premix, and was difficult to aspirate through and discharge from a micropipette. According to the present Example, it was made clear that a premix reagent containing 30 v/v % glycerol and 2.5 to 7.5 w/v % polyethylene glycol (glycerol concentration at the time of RT-PCR reaction: 6 v/v %, and polyethylene glycol concentration at the time of RT-PCR reaction: 0.5 to 1.5 w/v %) exhibited an excellent property as a 5-fold concentration premix reagent for one-step RT-PCR.

### (3) Study of Ethylene Glycol Concentration and Polyethylene Glycol Concentration-1

For the 5×premix having a glycerol concentration of 20 v/v % or 25 v/v % (glycerol concentration at the time of RT-PCR reaction: 4 v/v % or 5 v/v %, respectively) which was frozen under a storage condition of −30° C. in Example 6-(1), addition of ethylene glycol and polyethylene glycol was studied.

Each 2 mL of 20 kinds of 5×premixes containing a specific gravity-increasing agent at concentrations shown in Table 10 below, in addition to the composition shown in Table 6, were prepared. As a control, 2 mL of a 5×premix containing 2 v/v % glycerol and the components shown in Table 6 was prepared.

TABLE 10

| | Concentration of specific | | | Concentration of specific | | |
|---|---|---|---|---|---|---|
| | gravity-increasing agent in | | | gravity-increasing agent in RT- | | |
| | 5 × premix | | | PCR reaction solution | | |
| | Glycerol | PEG | EG | Glycerol | PEG | EG |
| | (v/v %) | (w/v %) | (v/v %) | (v/v %) | (w/v %) | (v/v %) |
| 1 | 20 | 0 | 0 | 4 | 0 | 0 |
| 2 | 20 | 0 | 2.5 | 4 | 0 | 0.5 |
| 3 | 20 | 0 | 5 | 4 | 0 | 1 |
| 4 | 20 | 0 | 7.5 | 4 | 0 | 1.5 |
| 5 | 20 | 0 | 10 | 4 | 0 | 2 |
| 6 | 20 | 5 | 0 | 4 | 1 | 0 |
| 7 | 20 | 5 | 2.5 | 4 | 1 | 0.5 |
| 8 | 20 | 5 | 5 | 4 | 1 | 1 |
| 9 | 20 | 5 | 7.5 | 4 | 1 | 1.5 |
| 10 | 20 | 5 | 10 | 4 | 1 | 2 |
| 11 | 25 | 0 | 0 | 5 | 0 | 0 |
| 12 | 25 | 0 | 2.5 | 5 | 0 | 0.5 |
| 13 | 25 | 0 | 5 | 5 | 0 | 1 |
| 14 | 25 | 0 | 7.5 | 5 | 0 | 1.5 |
| 15 | 25 | 0 | 10 | 5 | 0 | 2 |
| 16 | 25 | 5 | 0 | 5 | 1 | 0 |
| 17 | 25 | 5 | 2.5 | 5 | 1 | 0.5 |
| 18 | 25 | 5 | 5 | 5 | 1 | 1 |
| 19 | 25 | 5 | 7.5 | 5 | 1 | 1.5 |
| 20 | 25 | 5 | 10 | 5 | 1 | 2 |

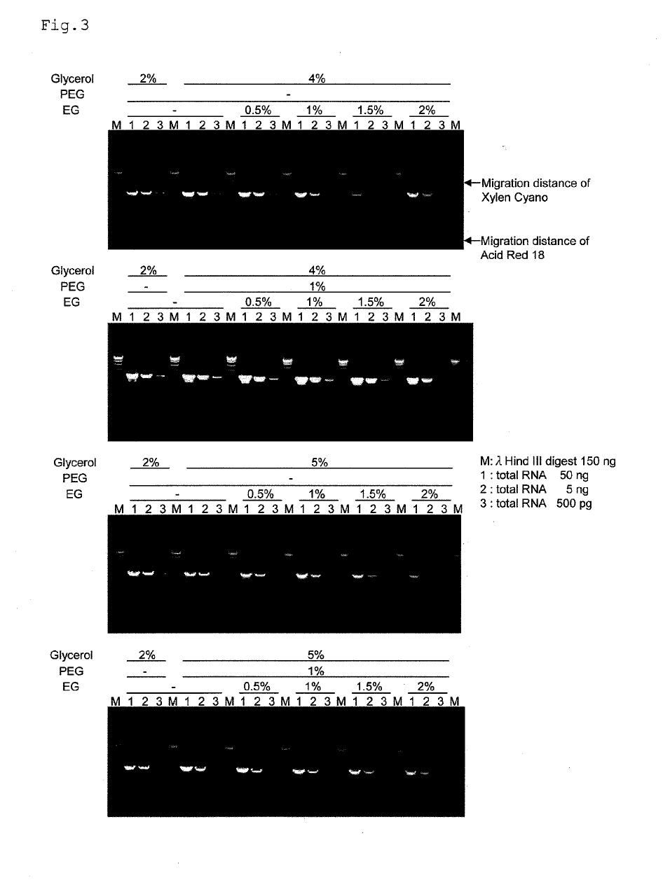

After each 650 μL of the prepared 5×premixes were dispensed into 1.5 mL tubes and allowed to stand for 1 to 2 days in a refrigerator (MEDICAL FREEZER) set at −30° C., the presence or absence of freezing was confirmed. Then, to each 5 μL of the 5×premixes were added 0.5 μL of 20 μM CCDN2F, 0.5 μL of 20 μM CCDN2R, 1 μL of a template total RNA solution obtained from HL60, and 18 μL of sterilized distilled water to prepare RT-PCR reaction solutions. At this time, as the template total RNA solution, the solutions of 50 ng/μL, 5 ng/μL, and 500 pg/μL were used for each 5×premix, and three kinds of reaction solutions containing different amounts of the template were prepared. In the prepared reaction solutions, one-step RT-PCR was carried out in the same manner as in Example 6-(2). The reaction solutions after completion of the reaction were subjected to 1% agarose electrophoresis, and the amount of an amplification product was confirmed by UV irradiation. The results are shown in Table 11. The meanings of symbols described in columns of operability, reactivity and comprehensive evaluation of Table 11 are as shown in Table 8. In addition, the results of agarose electrophoresis are shown in FIG. 3.

TABLE 11

| | | | Presence or | | | Compre- |
|---|---|---|---|---|---|---|
| Glycerol | PEG | EG | absence of | Opera- | Reac- | hensive |
| (v/v %) | (w/v %) | (v/v %) | freezing | bility | tivity | evaluation |
| 4 | 0 | 0 | ▪ | X | ◯ | X |
| | | 0.5 | ▪ | X | ◯ | X |
| | | 1 | ▪ | X | Δ | X |
| | | 1.5 | □ | ⊙ | Δ | ◯ |
| | | 2 | □ | ⊙ | Δ | ◯ |
| | 1 | 0 | ▪ | X | ◯ | X |
| | | 0.5 | ▪ | X | ◯ | X |
| | | 1 | □ | ⊙ | ◯ | ⊙ |
| | | 1.5 | □ | ⊙ | ◯ | ⊙ |
| | | 2 | □ | ⊙ | Δ | ◯ |
| 5 | 0 | 0 | ▪ | X | ◯ | X |
| | | 0.5 | □ | X | ◯ | X |
| | | 1 | □ | ⊙ | Δ | ◯ |
| | | 1.5 | □ | ⊙ | Δ | ◯ |
| | | 2 | □ | ⊙ | Δ | ◯ |
| | 1 | 0 | ▪ | X | ⊙ | X |
| | | 0.5 | □ | ⊙ | ◯ | ⊙ |
| | | 1 | □ | ⊙ | ◯ | ⊙ |
| | | 1.5 | □ | ⊙ | ◯ | ⊙ |
| | | 2 | □ | ⊙ | Δ | ◯ |

In the table, ▪ indicates that a sample was frozen at −30° C., and □ indicates that a sample was not frozen.

In Table 11, the concentration of glycerol, the concentration of polyethylene glycol, and the concentration of ethylene glycol indicate the concentrations in the reaction solutions for RT-PCR prepared so as to have 1-fold concentration. As shown in Table 11, it was made clear that even when a 5×premix had a glycerol concentration of less than 30 v/v % (glycerol concentration at the time of RT-PCR reaction: less than 6 v/v %), if polyethylene glycol and 5 to 7.5 v/v % ethylene glycol (ethylene glycol concentration at the time of RT-PCR reaction: 1 to 1.5 v/v %) coexisted, the 5×premix was not frozen even under a storage condition of −30° C. and could exhibit excellent reactivity of RT-PCR.

### (4) Study of Ethylene Glycol Concentration and Polyethylene Glycol Concentration-2

On the effect of the coexistence of glycerol, polyethylene glycol and ethylene glycol which was found in Example 6-(3), influence of the concentration of polyethylene glycol was confirmed.

Each 2 mL of 20 kinds of 5×premixes containing a specific gravity-increasing agent shown in Table 12 below in addition to the components shown in Table 6 were prepared. As a control, 2 mL of a 5×premix having 2 v/v % glycerol and the composition shown in Table 6 was prepared.

TABLE 12

| | Concentration of specific | | | Concentration of specific | | |
|---|---|---|---|---|---|---|
| | gravity-increasing agent in | | | gravity-increasing agent in RT- | | |
| | 5 × premix | | | PCR reaction solution | | |
| | Glycerol | PEG | EG | Glycerol | PEG | EG |
| | (v/v %) | (w/v %) | (v/v %) | (v/v %) | (w/v %) | (v/v %) |
| 1 | 20 | 2.5 | 5 | 4 | 0.5 | 1 |
| 2 | 20 | 2.5 | 7.5 | 4 | 0.5 | 1.5 |
| 3 | 20 | 3.75 | 5 | 4 | 0.75 | 1 |
| 4 | 20 | 3.75 | 7.5 | 4 | 0.75 | 1.5 |
| 5 | 20 | 5 | 5 | 4 | 1 | 1 |
| 6 | 20 | 5 | 7.5 | 4 | 1 | 1.5 |
| 7 | 20 | 7.5 | 5 | 4 | 1.5 | 1 |
| 8 | 20 | 7.5 | 7.5 | 4 | 1.5 | 1.5 |
| 9 | 25 | 2.5 | 5 | 5 | 0.5 | 1 |
| 10 | 25 | 2.5 | 7.5 | 5 | 0.5 | 1.5 |
| 11 | 25 | 3.75 | 5 | 5 | 0.75 | 1 |
| 12 | 25 | 3.75 | 7.5 | 5 | 0.75 | 1.5 |
| 13 | 25 | 5 | 5 | 5 | 1 | 1 |
| 14 | 25 | 5 | 7.5 | 5 | 1 | 1.5 |
| 15 | 25 | 7.5 | 5 | 5 | 1.5 | 1 |
| 16 | 25 | 7.5 | 7.5 | 5 | 1.5 | 1.5 |

After each 650 μL of the prepared 5×premixes were dispensed into 1.5 mL Eppendorf tubes and allowed to stand for 1 to 2 days in a refrigerator (MEDICAL FREEZER) set at −30° C., the presence or absence of freezing was confirmed. Then, in the same manner as in Example 6-(3), RT-PCR reaction solutions were prepared and one-step RT-PCR was carried out. The reaction solutions after completion of the reaction were subjected to 1% agarose electrophoresis, and the amount of an amplification product was confirmed by UV irradiation. The results are shown in Table 13. The meanings of symbols described in columns of operability, reactivity and comprehensive evaluation of Table 13 are as shown in Table 8.

TABLE 13

| | | | Presence or | | | Compre- |
|---|---|---|---|---|---|---|
| Glycerol | PEG | EG | absence of | Opera- | Reac- | hensive |
| (v/v %) | (w/v %) | (v/v %) | freezing | bility | tivity | evaluation |
| 4 | 0.5 | 1% | □ | ⊙ | ◯ | ⊙ |
| | | 1.5% | □ | ⊙ | ◯ | ⊙ |
| | 0.75 | 1% | □ | ⊙ | ◯ | ⊙ |
| | | 1.5% | □ | ⊙ | ◯ | ⊙ |
| | 1 | 1% | □ | ⊙ | ⊙ | ⊙ |
| | | 1.5% | □ | ⊙ | ⊙ | ⊙ |
| | 1.5 | 1% | □ | ⊙ | ⊙ | ⊙ |
| | | 1.5% | □ | ⊙ | ◯ | ⊙ |
| 5 | 0.5 | 1% | □ | ⊙ | ◯ | ⊙ |
| | | 1.5% | □ | ⊙ | ◯ | ⊙ |
| | 0.75 | 1% | □ | ⊙ | ◯ | ⊙ |
| | | 1.5% | □ | ⊙ | ◯ | ⊙ |
| | 1 | 1% | □ | ⊙ | ◯ | ⊙ |
| | | 1.5% | □ | ⊙ | ◯ | ⊙ |
| | 1.5 | 1% | □ | ⊙ | ⊙ | ⊙ |
| | | 1.5% | □ | ⊙ | ◯ | ⊙ |

In the table, ▪ indicates that a sample was frozen at −30° C., and □ indicates that a sample was not frozen.

In Table 13, the concentration of glycerol, the concentration of polyethylene glycol and the concentration of ethylene glycol indicate the concentrations in the reaction solutions for RT-PCR prepared so as to have 1-fold concentration. As shown in Table 13, it was made clear that even when a 5×premix had a glycerol concentration of 20 to 25 v/v % (glycerol concentration at the time of RT-PCR reaction: 4 to 5 v/v %), if 2.5 to 7.5 w/v % polyethylene glycol (polyethylene glycol concentration at the time of RT-PCR reaction: 0.5 to 1.5 w/v %) and 5 to 7.5 v/v % ethylene glycol (ethylene glycol concentration at the time of RT-PCR reaction: 1 to 1.5 v/v %) coexisted, the 5×premix was not frozen even under a storage condition of −30° C. and could exhibit excellent reactivity of RT-PCR.

### INDUSTRIAL APPLICABILITY

The present invention is useful in wide fields of biology, medical science, agriculture and the like.

### Sequence Listing Free Text

SEQ ID N0:1; Primer CCDN2F to amplify a 2.8 k by fragment of CCND2 gene.
SEQ ID NO:2; Primer CCND2R to amplify a 2.8 k by fragment of CCND2 gene.

1. A composition for a reverse transcription polymerase chain reaction, comprising a thermostable DNA polymerase, a reverse transcriptase, a dye marker, and a specific gravity-increasing agent.

2. The composition according to claim 1, wherein the specific gravity-increasing agent is selected from the group consisting of glycerol, ethylene glycol, polyethylene glycol and a combination thereof.

3. The composition according to claim 1, wherein the dye maker is selected from the group consisting of Tartrazine, Acid Red 18, Xylene Cyanol and a combination thereof.

4. The composition according to claim 2, which comprises 20 to 30% by volume of glycerol and 2.5 to 7.5% by weight/volume of polyethylene glycol as the specific gravity-increasing agents.

5. The composition according to claim 4, which further comprises 5 to 7.5% by volume of ethylene glycol as the specific gravity-increasing agent.

6. A reaction solution for a reverse transcription polymerase chain reaction, comprising the composition according to claim 1, as well as an RNA used as a template, and at least one kind of oligonucleotide primer.

7. The reaction solution according to claim 6, which comprises 4 to 6% by volume of glycerol and 0.5 to 1.5% by weight/volume of polyethylene glycol as the specific gravity-increasing agents.

8. The reaction solution according to claim 7, which further comprises 1 to 1.5% by volume of ethylene glycol as the specific gravity-increasing agent.

9. A method of synthesizing a cDNA, comprising a step of subjecting the reaction solution according to claim 6 to a reverse transcription polymerase chain reaction.

10. A method of detecting an RNA comprising steps of:
(A) subjecting the reaction solution according to claim 6 to a reverse transcription polymerase chain reaction, and
(B) detecting a cDNA amplified in the step (A) by electrophoresis.

11. A kit for a reverse transcription polymerase chain reaction, comprising an enzyme solution containing a thermostable DNA polymerase and a reverse transcriptase, as well as a reaction buffer containing a dye marker and a specific gravity-increasing agent.
